(19) United States
(12) Patent Application Publication
(10) Pub. No.: US 2022/0325227 A1
(43) Pub. Date: Oct. 13, 2022

(54) INTEGRATED FERMENTATION AND ELECTROLYSIS PROCESS FOR IMPROVING CARBON CAPTURE EFFICIENCY

(71) Applicant: LanzaTech, Inc., Skokie, IL (US)

(72) Inventors: Robert John Conrado, Washington, DC (US); Sean Dennis Simpson, Evanston, IL (US); Michael Emerson Martin, Chicago, IL (US); Jason Carl Bromley, Chicago, IL (US); Richard Russell Rosin, Glencoe, IL (US)

(21) Appl. No.: 17/658,566

(22) Filed: Apr. 8, 2022

Related U.S. Application Data
(60) Provisional application No. 63/173,262, filed on Apr. 9, 2021.

(51) Int. Cl. C12M 1/00; C12P 7/08; C25B 1/04; C25B 1/23; C25B 15/08; B01D 3/10; B01D 19/00

(52) U.S. Cl. CPC C12M 43/04; C12P 7/08; C25B 1/04; C25B 1/23; C25B 15/081; B01D 3/10; B01D 19/0068

(57) ABSTRACT

The disclosure provides for the integration of a fermentation process with at least one electrolysis process, a CO₂ to CO conversion unit, and a C1-generating industrial process. In particular, the disclosure provides process and a system for utilizing electrolysis products, for example H₂ and/or O₂ in a CO₂ to CO conversion unit to improve the process efficiency of at least one of the fermentation processes or the C1-generating industrial process. More particularly, the disclosure provides a process in which H₂ generated by electrolysis is passed to a CO₂ to CO conversion unit to improve the substrate efficiency for a fermentation process, and the O₂ generated by electrolysis process is used to improve the composition of the C1-containing tail gas generated by the C1-generating industrial process.

### CROSS REFERENCE TO RELATED APPLICATIONS

This application claims the benefit of U.S. Provisional Patent Application No. 63/173,262, filed Apr. 9, 2021, the entirety of which is incorporated herein by reference.

### FIELD

The disclosure relates to an integrated fermentation and industrial process and an apparatus for improving carbon capture efficiency.

### BACKGROUND

Carbon dioxide (CO₂) accounts for about 76% of global greenhouse gas emissions from human activities, with methane (16%), nitrous oxide (6%), and fluorinated gases (2%) accounting for the balance (United States Environmental Protection Agency). The majority of CO₂ comes from the burning fossil fuels to produce energy, although industrial and forestry practices also emit CO₂ into the atmosphere. Reduction of greenhouse gas emissions, particularly CO₂, is critical to halt the progression of global warming and the accompanying shifts in climate and weather.

It has long been recognized that catalytic processes, such as the Fischer-Tropsch process, may be used to convert gases containing carbon dioxide (CO₂), carbon monoxide (CO), and/or hydrogen (H₂) into a variety of fuels and chemicals. Recently, however, gas fermentation has emerged as an alternative platform for the biological fixation of such gases. In particular, anaerobic C1-fixing microorganisms have been demonstrated to convert gases containing CO₂, CO, and/or H₂ into products, like ethanol and 2,3-butanediol.

Such gasses may be derived, for example, from industrial processes, including gas from carbohydrate fermentation, gas from cement making, pulp and paper making, ferrous or non-ferrous metal products manufacturing, steel making, oil refining and associated processes, petrochemical production, electric power production, carbon black production, ammonia production, methanol production, coke production, anaerobic or aerobic digestion, synthesis gas (derived from sources including but not limited to biomass, liquid waste streams, solid waste streams, municipal streams, fossil resources including natural gas, coal and oil), natural gas extraction, oil extraction, metallurgical processes, for production and/or refinement of aluminium, copper, and/or ferroalloys, geological reservoirs, and catalytic processes (derived from steam sources including but not limited to steam methane reforming, steam naphtha reforming, petroleum coke gasification, catalyst regeneration, fluid catalyst cracking, catalyst regeneration-naphtha reforming, and dry methane reforming).

However, efficient production of fermentation products may be limited due to several factors for example, by slow microbial growth, limited gas uptake, sensitivity to toxins, or diversion of carbon substrates into undesired by-products. Therefore, these industrial gasses may require treatment or re-composition to be optimized for use in gas fermentation systems. In particular, industrial gasses may lack sufficient amounts of H₂ to drive net fixation of CO₂ by gas fermentation and reduce CO₂ emissions to the atmosphere. For example, much of the demand for hydrogen in industry is met by methane steam reforming. Conventionally, this reaction results in the production of CO and H₂ with little CO₂ as a by-product. The carbon monoxide is then reacted in one, or a series of two, water gas shift reactors to further generate H₂ and CO₂. Hydrogen is then purified in a pressure swing adsorption (PSA) unit. A purified hydrogen stream and a PSA tail gas comprising some hydrogen and unreacted CO₂ and CO are produced by the PSA unit. The PSA tail gas often has too little CO to be used directedly as a feed to gas fermentation. One technique to increase the CO concentration in the PSA tail gas involves utilizing only a high temperature water gas shift reactor. However, without an additional low temperature water gas shift reactor, the amount of purified hydrogen produced is less. Some refineries cannot suffer this loss of purified hydrogen in the purified hydrogen stream.

High hydrogen streams are beneficial to fermentation products which have low energy demand and where CO₂ may be used as a reactant, such as with ethanol production. A need exists for a process and system to maintain the high yield of purified hydrogen and yet provide a feed to gas fermentation having a suitable concentration of CO. Accordingly, there remains a need for improved integration of industrial processes with gas fermentation systems, including processes for enriching the H₂ content of industrial gases delivered to gas fermentation systems.

### SUMMARY

The disclosure provides a process for improving carbon capture in an integrated fermentation and industrial process. The process comprises obtaining a first gas stream comprising O₂ and a second gas stream comprising CO from a CO₂ electrolysis unit. A third gas stream comprising H₂ is obtained from H₂O electrolysis. At least a portion of the first gas stream is passed in an industrial process wherein a tail gas stream comprising CO₂ is produced. At least a portion of the tail gas stream and at least a portion of the third gas stream are passed to a CO₂ to CO conversion system to produce a gaseous feed stream comprising CO. The gaseous feed stream, the second gas stream, optionally at least a portion of the third gas stream, and optionally at least a portion of the tail gas stream are passed to a gas fermentation bioreactor comprising a culture of at least one C1-fixing microorganism. The culture is fermented to produce at least one fermentation product and an exit gas stream comprising CO₂ which is recycled to CO₂ electrolysis process.

The industrial process is selected from the group consisting of selected from a partial oxidation process, a gasification process, and a complete oxidation process. The CO₂ electrolysis and/or H₂O electrolysis process requires an energy input, and the energy input may be derived from a renewable energy source. At least a portion of the tail gas stream may be passed to a treatment unit to generate a treated tail gas stream. The treated tail gas stream may be recycled to the CO₂ electrolysis unit. The CO₂ to CO conversion system is at least one selected from reverse water gas reaction system, a thermo-catalytic conversion system, partial combustion system, or plasma conversion system. The at least one C1 fixing bacterium is selected from Clostridium autoethanogenum, Clostridium ljungdahlii, or Clostridium ragsdalei. The fermentation product(s) is selected from the group consisting of ethanol, acetate, butanol, butyrate, 2,3-butanediol, lactate, butene, butadiene, methyl ethyl ketone, ethylene, acetone, isopropanol, lipids, 3-hydroxypropionate, isoprene, fatty acids, 2-butanol, 1,2-propanediol, and 1-propanol.

The disclosure further provides an integrated system for producing one or more fermentation products, the system comprising: a CO₂ electrolysis unit having a first gas stream conduit and a second gas stream conduit; an industrial process zone in fluid communication with the first gas conduit, having a tail gas conduit; H₂O electrolysis unit having a third gas stream conduit; a CO₂ to CO conversion system in fluid communication with the tail gas conduit and with the third gas stream conduit, having a feed stream conduit; and a gas fermentation bioreactor unit in fluid communication with the feed stream conduit, with the second gas stream conduit, and with the third gas stream conduit, having a product stream conduit.

In one embodiment, the CO₂ electrolysis unit and/or H₂O electrolysis unit is further in communication with a renewable energy production unit. The CO₂ to CO conversion system is selected from reverse water gas reaction system, a thermo-catalytic conversion system, partial combustion system, or plasma conversion system.

In one embodiment, the system further comprising a treatment unit in fluid communication with the tail gas conduit and the CO₂ electrolysis unit. The gas fermentation bioreactor unit further in fluid communication with the third gas stream conduit, the tail gas conduit and having an exit gas stream conduit. The exit gas stream conduit is in fluid communication with the CO₂ electrolysis unit.

The integrated system has the benefit of producing a valuable carbon containing product from a C1 waste gas and reducing CO₂ emissions. The provision of an electrolyzer for the electrolysis of water or carbon dioxide also reduces the requirement for air separation by alternative means, as O₂ produced by the electrolysis process can replace or supplement O₂ requirements of the industrial process. The industrial process zone is selected from a partial oxidation process zone, a gasification process zone, and a complete oxidation process zone.

The disclosure further provides an integrated fermentation and industrial process. The process comprises: obtaining a first gas stream comprising CO and H₂, a second gas stream comprising CO₂ and a third gas stream comprising H₂ from one or more industrial processes. An energy input is passed to a H₂O electrolysis unit to obtain a fourth gas stream comprising H₂ and a fifth gas stream comprising O₂. A first portion of the first gas stream, and a first portion of the second gas stream are passed to a first gas treatment unit, and a first portion of the third gas stream is passed to a second gas treatment unit to obtain a treated first gas stream, a treated second gas stream and a treated third gas stream. A second portion of the second gas stream, the treated second gas stream, a second portion of the third gas stream, the treated third gas stream, a first portion of the fourth gas stream and optionally a first portion of the treated first gas stream are passed to a CO₂ to CO conversion system to produce a gaseous feed stream comprising CO and an output stream comprising H₂O. The output stream is passed to the H₂O electrolysis unit. Optionally the gaseous feed stream is passed to a third gas treatment unit to obtain a treated gaseous feed stream. The treated gaseous feed stream, a second portion of the first gas stream, a second portion of the treated first gas stream, optionally a second portion of the third gas stream and optionally a second portion of the fourth gas stream are passed to a gas fermentation bioreactor unit to produce a gas fermentation stream and a tail gas comprising H₂. The gas fermentation stream is passed to a degasser unit to obtain a product stream comprising at least one fermentation product and CO₂. A first portion of the product stream is passed to a vacuum distillation unit to separate into at least one fermentation product and an exit gas stream comprising CO₂. A second portion of the product stream is passed to the first gas treatment unit and optionally a third portion of the product stream is passed to the CO₂ to CO conversion system. The exit stream is passed to the gas fermentation bioreactor unit. A first portion of the tail gas stream is passed to the second gas treatment unit and optionally a second portion of the tail gas stream is passed to the CO₂ to CO conversion system. A third portion of the tail gas stream and fifth gas stream are passed to an oxidizer unit.

In one embodiment the industrial process is selected from a syngas emitting industrial process, a CO₂ emitting industrial process and a H₂ emitting industrial process. In an embodiment, the industrial process is selected from carbohydrate fermentation, gas fermentation, cement making, pulp and paper making, steel making, oil refining, petrochemical production, coke production, anaerobic digestion, aerobic digestion, natural gas extraction, oil extraction, geological reservoirs, metallurgical processes, refinement of aluminium, copper and or ferroalloys, for production of aluminium, copper, and or ferroalloys, or any combination thereof; or the synthesis gas process is selected from gasification of gasification of coal, gasification of refinery residues, gasification of biomass, gasification of lignocellulosic material, black liquor gasification, gasification of municipal solid waste, gasification of industrial solid waste, gasification of sewerage, gasification of sludge from wastewater treatment, reforming of natural gas, reforming of biogas, reforming of landfill gas or any combination thereof.

In a one embodiment, energy input for the electrolyzer is provided by a renewable energy production zone. The first gas treatment unit, the second gas treatment unit and the third gas treatment unit comprise a sulfur removal module. The CO₂ to CO conversion system is at least one unit selected from reverse water gas reaction system, a CO₂ electrolysis system, a thermo-catalytic conversion system, electro-catalytic conversion system, partial combustion system and plasma conversion system. The oxidizer unit is selected from a thermal oxidizer unit, a thermal reformer unit, a combined heat and power unit or a syngas generation unit.

### BRIEF DESCRIPTION OF THE DRAWINGS

FIG. 1 shows a process integration scheme depicting integration of an industrial process with a fermentation process and a carbon dioxide and water electrolysis process, and a CO₂ to CO conversion system, according to one embodiment.

FIG. 2 shows a schematic process for the integration of a cement production process with an electrolysis process and a gas fermentation process, in accordance with one embodiment of the disclosure.

FIG. 3 shows a process integration scheme depicting integration of one or more industrial processes with a CO₂ to CO conversion system, electrolysis unit(s) and a gas fermentation process, in accordance with one embodiment of the disclosure.

### DETAILED DESCRIPTION

Disclosed is a process for improving carbon capture efficiency in an integrated fermentation and industrial process. The integration of a C1-generating industrial process, H₂-emitting industrial process with a C1-fixing fermentation process, a CO₂ to CO conversion system and electrolysis processes provides substantial benefits to both the C1-generating industrial process and the C1-fixing fermentation process. “C1” refers to a one-carbon molecule, for example, CO, CO₂, CH₄, or CH₃OH.

A “C1-generating industrial process” is an industrial process which generates at least one C1-containing gas during its operation process. The C1-generating industrial process is intended to include any industrial process which generate a C1-containing gas as either a desired end product, or as a by-product in the production of one or more desired end products. Exemplary C1-generating industrial processes include, but are not limited to, steel manufacturing process, including basic oxygen furnace (BOF) processes; steel making processes, blast furnace (BF) processes and coke oven gas processes, gasification processes, including, gasification of municipal solid waste, biomass gasification, pet coke gasification and coal gasification, titanium dioxide production processes, cement production processes, natural gas power processes, and coal fired power processes. The C1-generating industrial process may further include traditional biomass-to-ethanol fermentation processes involving the conversion of sugars derived from biomass feedstocks to ethanol. Suitable biomass feedstocks for the traditional ethanol fermentation process include corn fiber, corn stover, bagasse, and rice straw.

A “desired end product” is intended to encompass the primary or target product of the industrial process. For example, the desired end product of a steel manufacturing process is a steel product, and a C1-containing gas is generated as a by-product, however in a MSW gasification process, syngas, a C1-containing gas is the desired end product of the gasification process.

The disclosure provides an integrated C1-generating industrial process and a C1-fixing fermentation process coupled with H₂O and/or CO₂ electrolysis process, and CO₂ to CO conversion process, to improve the composition of C1-containing gases generated by the industrial process. C1-fixing fermentation process provides a platform for the biological fixation of C1-containing gases using C1-fixing microorganisms. In particular, C1-fixing microorganisms convert C1-containing gases and/or H₂ into products such as ethanol and 2,3-butanediol. The present disclosure provides processes and systems for substantially reducing the total amount of CO₂ emitted from an integrated facility.

Hydrogen is a suitable source of energy for fermentation processes. Hydrogen may be used to improve the fermentation substrate composition. Hydrogen provides energy required by the microorganism to convert carbon containing gases into useful products. When optimal concentrations of hydrogen are provided, the microbial culture can produce the desired fermentation products (i.e., ethanol) with little co-production of carbon dioxide.

Hydrogen may be produced by H₂O electrolysis process, defined by the following stoichiometric reaction: 2H₂O+electricity→2H₂+O₂+heat. Water electrolysis technologies are known, and exemplary processes include alkaline water electrolysis, protein exchange membrane (PEM) electrolysis, and solid oxide electrolysis. Suitable electrolyzers include Alkaline electrolyzers, PEM electrolyzers, and solid oxide electrolyzers The Hydrogen produced by electrolysis may be used as a feedstock for gas fermentation when supplied in combination with industrial waste gases containing a suitable carbon source e.g., at least one C1 containing gas, such as Carbon monoxide (CO) and/or Carbon dioxide (CO₂).

Electrolysis processes and electrolyzers for the reduction of CO₂ are known. The use of different catalysts for CO₂ reduction impact the end product. Catalysts including Au, Ag, Zn, Pd, and Ga catalysts have been shown effective for the production of CO from CO₂. Standard electrolyzers, such as those described above for water electrolysis may be used. Carbon monoxide produced by CO₂ electrolysis may be used as a feedstock for gas fermentation.

Additionally, the produced CO may be blended with an industrial gas stream, as additional feedstock supply. CO₂ and an energy input may produce carbon monoxide and O₂ by the CO₂ electrolysis process, defined by the following stoichiometric reaction: 2CO₂+electricity→2 CO+O₂+heat.

The energy input for the H₂O electrolysis unit or CO₂ electrolysis unit may be derived from a renewable energy source. Exemplary sources for the renewable energy include, but are not limited to wind power, hydropower, solar energy, geothermal energy, nuclear energy, and combinations thereof.

Carbon monoxide produced by electrolysis of CO₂ in a CO₂ electrolysis unit may be used to improve the fermentation substrate composition and can enrich the CO content of the industrial waste gas being utilized as a fermentation substrate. Additionally, CO₂ produced by the fermentation process may be recycled as a feedstock for the CO₂ electrolyzer, thereby further reducing CO₂ emissions and increasing the amount of carbon captured in liquid fermentation products.

In a number of industrial processes, oxygen is sourced from an air feed. In partial oxidation processes, such as basic oxygen furnace (BOF) processes; steel making processes, blast furnace (BF) processes, titanium dioxide production processes, ferroalloy production processes and gasification processes, O₂ is typically produced from air using an air separation process, such as cryogenic distillation or PSA separation. According to the present disclosure, O₂ produced by the electrolysis process, can reduce, or replace the requirement for air separation.

O₂ produced as a by-product of the electrolysis processes provides additional benefit to the use of industrial gas for fermentation. While the fermentation processes of the disclosure are anaerobic processes, the O₂ by-product of the both the H₂O electrolysis and CO₂ electrolysis process may be used in the C1-generating industrial process from which the C1-containing tail gas is obtained. The high-purity O₂ by-product of the electrolysis process may be integrated with the industrial process and beneficially offset costs and in some cases have synergy that further reduces costs for both the industrial process as well as the subsequent gas fermentation. Typically, the industrial processes derive the required oxygen by air separation. Production of oxygen by air separation is an energy intensive process which involves cryogenically separating O₂ from N₂ to achieve the highest purity. Co-production of O₂ by electrolysis, and displacing O₂ produced by air separation, could offset up to 5% of the electricity costs in an industrial process.

Electrolysis products such as hydrogen, carbon monoxide and oxygen can also be utilized to improve overall efficiency of the integration of industrial production processes and gas fermentation processes such as in industrial processes where the C1-containing tail gas is suitable for use as a fermentation substrate, further substrate optimisation by blending with hydrogen or carbon monoxide can improve the over-all carbon utilisation of the fermentation. Efficiency may be improved by (i) using hydrogen to improve the fermentation substrate composition; (ii) using carbon monoxide to improve the fermentation substrate composition; (iii) using oxygen derived from the electrolysis process to offset the oxygen requirements of the industrial process; (iv) recycling CO₂ from the fermentation process exit gas stream to a CO₂ electrolyzer to produce additional CO and further reduce CO₂ emissions; or (v) any combination of the above.

The integrated process of the present disclosure comprises obtaining a first gas stream comprising O₂ and a second gas stream comprising CO from a CO₂ electrolysis unit. A third gas stream comprising H₂ is obtained from H₂O electrolysis unit. At least a portion of the first gas stream is converted to a tail gas stream comprising CO₂ in an industrial process zone. At least a portion of the tail gas stream and optionally at least a portion of the third gas stream are passed to a CO₂ to CO conversion system to produce a gaseous feed stream comprising CO.

The CO₂ to CO conversion system is at least one unit selected from reverse water gas reaction system, CO₂ electrolysis system, thermo-catalytic conversion system, electro-catalytic conversion system, partial combustion system, plasma conversion system, or any combination thereof. The reverse water gas reaction unit (rWGR) produces water from carbon dioxide and hydrogen, with carbon monoxide as a side product. The reverse water gas reaction unit may comprise a single stage or more than one stage. The different stages may be conducted at different temperatures and may use different catalysts. The thermo-catalytic conversion disrupts the stable atomic and molecular bonds of CO₂ and other reactants over a catalyst by using thermal energy as the driving force of the reaction to produce CO. Since CO₂ molecules are thermodynamically and chemically stable, if CO₂ is used as a single reactant, large amounts of energy are required. Therefore, often other substances such as hydrogen are used as a co-reactant to make the thermodynamic process easier. Many catalysts are known for the process such as metals and metal oxides as well as nano-sized catalyst metal-organic frameworks. Various carbon materials have been employed as carriers for the catalysts. The electro-catalytic conversion is the electrocatalytic reduction of carbon dioxide to produce synthesis gas from water and carbon dioxide. Such electro-catalytic conversion, also referred to as electrochemical conversion, of carbon dioxide typically involves electrodes in an electrochemical cell having a solution supporting an electrolyte through which carbon dioxide is bubbled, see for example U.S. Pat. No. 10,119,196. The synthesis gas, also known as syngas, produced comprises CO, and is separated from the solution of the electrochemical cell and removed. The combination of photocatalysis and electrocatalysis in photoelectrocatalysis which uses for example sunlight irradiation is also a suitable variation.

The gaseous feed stream, the second gas stream, and optionally at least a portion of the third gas stream are passed to a gas fermentation bioreactor comprising a culture of at least one C1-fixing microorganism. The culture is fermented to produce at least one fermentation product and an exit gas stream comprising CO₂ which is recycled to CO₂ electrolysis process. When discussing recycling herein, the description of recycling or passing a stream to a unit is mean to include direct independent introduction of the stream to the unit, or combination of the stream with another input to the unit.

The gas fermentation bioreactor may be a fermentation system consisting of one or more vessels and/or towers or piping arrangements. Examples of the gas fermentation bioreactors include continuous stirred tank reactor (CSTR), immobilized cell reactor (ICR), trickle bed reactor (TBR), bubble column, gas lift fermenter, static mixer, circulated loop reactor, membrane reactor, such as hollow fibre membrane bioreactor (HFM BR), or other device suitable for gas-liquid contact. The gas fermentation may comprise multiple reactors or stages, either in parallel or in series. The gas fermentation bioreactor may be a production reactor, where most of the fermentation products are produced.

The gas fermentation bioreactor includes a culture of one or more C1-fixing microorganisms that have the ability to produce one or more products from a C1-carbon source. “C1” refers to a one-carbon molecule, for example, CO or CO₂. “C1-carbon source” refers a one carbon-molecule that serves as a partial or sole carbon source for the microorganism. For example, a C1-carbon source may comprise one or more of CO, CO₂, or CH₂O₂. In some embodiments, the C1-carbon source may comprise one or both of CO and CO₂. Typically, the C1-fixing microorganism is a C1-fixing bacterium. In an embodiment, the microorganism is derived from a C1-fixing microorganism identified in Table 1. The microorganism may be classified based on functional characteristics. For example, the microorganism may be derived from a C1-fixing microorganism, an anaerobe, an acetogen, an ethanologen, and/or a carboxydotroph. Table 1 provides a representative list of microorganisms and identifies their functional characteristics.

TABLE 1

| | C1-fixing | Anaerobe | Acetogen | Ethanologen | Autotroph | Carboxydotroph | Methanotroph |
|---|---|---|---|---|---|---|---|
| Acetobacterium woodii | + | + | + | +/− ¹ | − | +/− ² | − |
| Alkalibaculum bacchii | + | + | + | + | + | + | − |
| Blautia product | + | + | + | − | + | + | − |
| Butyribacterium methylotrophicum | + | + | + | + | + | + | − |
| Clostridium aceticum | + | + | + | − | + | + | − |
| Clostridium autoethanogenum | + | + | + | + | + | + | − |
| Clostridium carboxidivorans | + | + | + | + | + | + | − |
| Clostridium coskatii | + | + | + | + | + | + | − |
| Clostridium drakei | + | + | + | − | + | + | − |
| Clostridium formicoaceticum | + | + | + | − | + | + | − |
| Clostridium ljungdahlii | + | + | + | + | + | + | − |
| Clostridium magnum | + | + | + | − | + | +/− ³ | − |
| Clostridium ragsdalei | + | + | + | + | + | + | − |
| Clostridium scatologenes | + | + | + | − | + | + | − |
| Eubacterium limosum | + | + | + | − | + | + | − |
| Moorella thermautotrophica | + | + | + | + | + | + | − |
| Moorella thermoacetica | + | + | + | − ⁴ | + | + | − |
| (formerly | | | | | | | |
| Clostridium thermoacelicum) | | | | | | | |
| Oxobacter pfennigii | + | + | + | − | + | + | − |
| Sporomusa ovata | + | + | + | − | + | +/− ⁵ | − |
| Sporomusa silvacetica | + | + | + | − | + | +/− ⁶ | − |
| Sporomusa sphaeroides | + | + | + | − | + | +/− ⁷ | − |
| Thermoanaerobacter kivui | + | + | + | − | + | − | − |

An “anaerobe” is a microorganism that does not require oxygen for growth. An anaerobe may react negatively or even die if oxygen is present above a certain threshold. Typically, the microorganism is an anaerobe. In an embodiment, the microorganism is or is derived from an anaerobe identified in Table 1.

An “acetogen” is a microorganism that produces or is capable of producing acetate or acetic acid as a product of anaerobic respiration. Typically, acetogens are obligately anaerobic bacteria that use the Wood-Ljungdahl pathway as their main mechanism for energy conservation and for synthesis of acetyl-CoA and acetyl-CoA-derived products, such as acetate. All naturally occurring acetogens are C1-fixing, anaerobic, autotrophic, and non-methanotrophic.

The microorganism may be a member of the genus Clostridium. In one embodiment, the microorganism is obtained from the cluster of Clostridia comprising the species Clostridium autoethanogenum, Clostridium ljungdahlii, and Clostridium ragsdalei.

The microorganism of the disclosure may be cultured to produce one or more products. For instance, Clostridium autoethanogenum produces or may be engineered to produce ethanol (WO 2007/117157), acetate (WO 2007/117157), butanol (WO 2008/115080 and WO 2012/053905), butyrate (WO 2008/115080), 2,3-butanediol (WO 2009/151342), lactate (WO 2011/112103), butene (WO 2012/024522), butadiene (WO 2012/024522), methyl ethyl ketone (2-butanone) (WO 2012/024522 and WO 2013/185123), ethylene (WO 2012/026833), acetone (WO 2012/115527), isopropanol (WO 2012/115527), lipids (WO 2013/036147), 3-hydroxypropionate (3-HP) (WO 2013/180581), isoprene (WO 2013/180584), fatty acids (WO 2013/191567), 2-butanol (WO 2013/185123), 1,2-propanediol (WO 2014/0369152), 1-propanol (WO 2014/0369152), ethylene glycol (WO 2019/125400), and 2-phenylethanol (WO 2021/188190). In addition to one or more target products, the microorganism of the disclosure may also produce ethanol, acetate, and/or 2,3-butanediol. In certain embodiments, microbial biomass itself may be considered a product.

The culture is generally maintained in an aqueous culture medium that contains nutrients, vitamins, and/or minerals sufficient to permit growth of the microorganism. The aqueous culture medium may be an anaerobic microbial growth medium, such as a minimal anaerobic microbial growth medium. Suitable media are well known in the art.

The culture and/or fermentation may be carried out under appropriate conditions for production of the target product. The culture/fermentation may be performed under anaerobic conditions. Reaction conditions to consider include pressure or partial pressure, temperature, gas flow rate, liquid flow rate, media pH, media redox potential, agitation rate if using a continuous stirred tank reactor, inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting, since products may be consumed by the culture under gas-limited conditions.

Operating a gas fermentation bioreactor at elevated pressures allows for an increased rate of gas mass transfer from the gas phase to the liquid phase. Accordingly, the culture fermentation may be performed at pressures higher than atmospheric pressure. Also, since a given gas conversion rate is in part a function of the substrate retention time, the conversion rate dictates the required volume of a gas fermentation bioreactor. The use of pressurized systems can greatly reduce the volume of the gas fermentation bioreactor required and, consequently, the capital cost of the culture/fermentation equipment. Accordingly, the retention time, defined as the liquid volume in the gas fermentation bioreactor divided by the input gas flow rate, may be reduced when the gas fermentation bioreactors are maintained at elevated pressure rather than atmospheric pressure. The optimum reaction conditions will depend partly on the particular microorganism used. However, in general, the fermentation may be operated at a pressure higher than atmospheric pressure.

Target products may be separated from the fermentation broth using any method or combination of methods known in the art, including, for example, fractional distillation, evaporation, pervaporation, gas stripping, phase separation, extractive separation, including for example, liquid-liquid extraction. In certain embodiments, target products are recovered from the fermentation broth by continuously removing a portion of the broth from the gas fermentation bioreactor, separating microbial cells from the broth and separating the target product from the aqueous remainder. Alcohols, acetone and/or other by-products may be recovered, for example, by distillation. Acids may be recovered, for example, by adsorption on activated charcoal. Separated microbial biomass may be recycled to the gas fermentation bioreactor. The solution remaining after the target products have been removed may also be recycled to the gas fermentation bioreactor. Additional nutrients may be added to the recycled solution to replenish the medium before it is returned to the gas fermentation bioreactor.

In some instances, the gas compositions of the C1 containing gases are not ideal for a typical fermentation process. Due to geological restrictions, lack of available hydrogen sources, or cost consideration, the use hydrogen for fermentation processes has been challenging. By utilizing renewable hydrogen (e.g., hydrogen produced by electrolysis), a number of these restrictions may be reduced or removed. Furthermore, blending C1 containing gas with a renewable hydrogen stream, provides an energetically improved blended substrate stream.

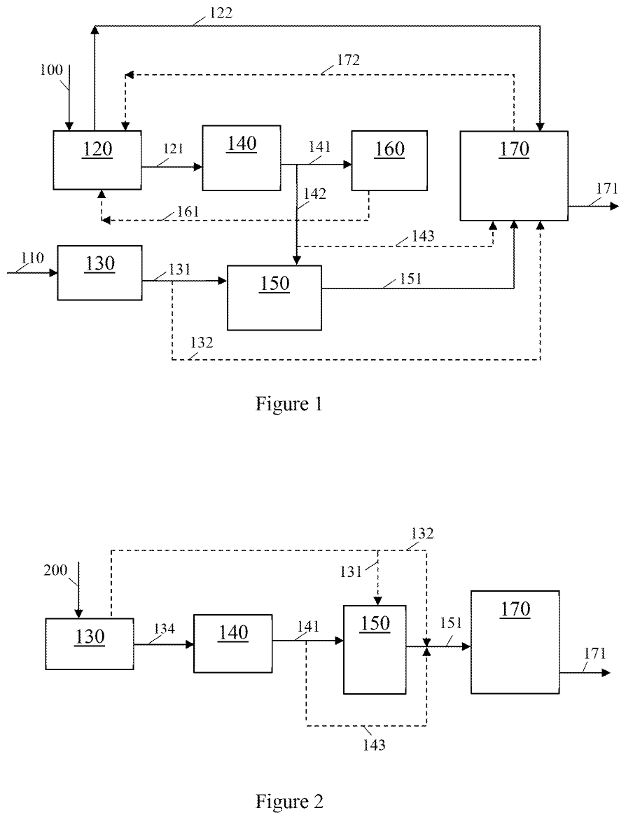

Some embodiments of the disclosure may be described by reference to the process configuration shown in FIGS. 1 to 3, which relate to both apparatus and methods to carry out the disclosure. Any reference to a method “step” includes reference to an apparatus “unit” or equipment that is suitable to carry out the step, and vice-versa. The Figures have been simplified by the deletion of a large number of apparatuses customarily employed in a process of this nature, such as vessel internals, temperature and pressure controls systems, flow control valves, recycle pumps, etc. which are not specifically required to illustrate the performance of the disclosure.

FIG. 1 depicts an integrated system having a fermentation process, a carbon dioxide and water electrolysis process with a CO₂ to CO conversion system and process for the production of at least one fermentation product from a gaseous stream in accordance with one embodiment of the disclosure. CO₂ electrolysis unit 120 receives renewable energy input 100. Exemplary sources for the renewable energy input include, but are not limited to, wind power, hydropower, solar energy, geothermal energy, nuclear energy, and combinations thereof. A first gas stream comprising O₂ and a second gas stream comprising CO may be obtained from the CO₂ electrolysis unit 120. The first gas stream 121 is passed to industrial process unit 140, to displace air requirements of the industrial process unit 140, and the industrial process produces a tail gas stream 141 comprising CO₂. At least a portion tail gas stream 141 may be passed to gas treatment unit 160. Gas treatment unit 160 comprises at least one gas treatment module for removal of one or more contaminants from tail gas stream 141 generate treated tail gas stream 161 which may be passed to CO₂ electrolysis unit 120. Second gas stream 122 comprising CO is passed to gas fermentation bioreactor unit 170 comprising a culture of at least one C1-fixing microorganism. H₂ electrolysis unit 130 receives renewable energy input 110 to produce a third gas stream 131 comprising H₂. At least a portion 142 of the tail gas stream 141 and at least a portion of the third gas stream 131 are passed to CO₂ to CO conversion system 150 to produce gaseous feed stream 151 comprising CO. Gaseous feed stream 151 is passed to the gas fermentation bioreactor unit 170. Optionally, at least a portion of tail gas stream 143 and optionally at least a portion 132 of the third gas stream 131 may be passed to the gas fermentation bioreactor unit 170. The culture is fermented to produce one or more fermentation products 171 and an exit gas stream 172 comprising CO₂. The exit gas stream 172 may be recycled to CO₂ electrolysis unit 120.

In one embodiment, industrial process unit 140 is selected from a partial oxidation process unit, a gasification process unit, a complete oxidation process unit, or any combination thereof. A partial oxidation process is an industrial process comprising a partial oxidation reaction. The partial oxidation process may be selected from a basic oxygen furnace (BOF) reaction, a COREX or FINEX steel making process, a blast furnace (BF) process, a ferroalloy process, a titanium dioxide production process, a gasification process, or any combination thereof. The gasification process may be selected from a municipal solid waste gasification process, a biomass gasification process, a pet coke gasification process, a coal gasification process, or any combination thereof. At least one of the fermentation products in stream 171 may be ethanol, butyrate, 2,3-butanediol, lactate, butene, butadiene, methyl ethyl ketone, ethylene, acetone, isopropanol, lipids, 3-hydroypropionate, terpenes, fatty acids, 2-butanol, 1,2-propanediol, 1-propanol, ethylene glycol, or any combination thereof.

Tail gas stream from the industrial process comprises at least one C1-component. The C1-component in the C1-containing tail gas is selected from carbon monoxide, carbon dioxide, methane, or combinations thereof. The C1-containing tail gas may further comprise one or more non-C1 components, such as nitrogen and hydrogen. The C1-containing tail gas may further comprise contaminant components from the industrial process. In an embodiment, the C1-containing tail gas is passed to a gas treatment unit for the removal of at least one contaminant or non-C1 component, to provide a purified C1-containing tail gas, prior to being passed to the gas fermentation bioreactor.

A number of industrial processes produce C1 containing gases, which may not be ideal for a typical C1 fermentation processes, and such industrial processes may include cement production processes, natural gas power plants, refinery processes, ethanol producing fermentation processes, or any combination thereof. Cement production process typically produce CO₂ rich exit gas streams. CO₂ may be utilised by C1-fixing microorganisms, however hydrogen is typically employed as well to provide the energy needed for fixing CO₂ into products.

The integration of a complete oxidation process, such as a cement production process, with a CO₂ and/or H₂O electrolyzer units, a CO₂ to CO conversion system and a C1-fixing fermentation process provides a number of synergistic benefits including (i) providing a mechanism for converting CO₂ to CO; (ii) O₂ provided by the electrolysis process displaces the air feed to the cement production process with and increases the composition of CO₂ in the exit gas of the cement production process; (iii) CO₂ produced by the fermentation process may be recycled to the CO₂ electrolyzer and converted to CO substrate for fermentation, thereby further decreasing CO₂ emissions by the combined processes.

FIG. 2 shows a schematic process for the integration of a cement production process with an electrolysis process and a gas fermentation process. A first gas stream 132 comprising H₂ and a second gas stream 134 comprising O₂ are produced by electrolysis of water stream 200 using a renewable energy input, in a water electrolysis unit 130. Second gas stream 134 is passed to a cement production unit 140, to displace at least a portion of the typical air requirement of the cement production process. Cement production process 140 produces CO₂ rich tail gas stream 141. A first portion of the CO₂ enriched tail gas stream 141, and optionally a first portion of the first gas stream 131 are passed to a CO₂ to CO conversion system 150 to produce an exit gas stream comprising CO. Optionally a second portion of the CO₂ enriched tail gas stream 143 and a second portion of the first gas stream 132 may be combined with exit gas stream to provide a C1-containing feed stream 151. The C1-containing feed stream 151 is passed to gas fermentation bioreactor 170 containing a culture of C1-fixing bacteria. C1-containing feed stream 151 is fermented to produce at least one fermentation product stream 171.

In an embodiment, the integration of a cement production process with a water electrolysis process enables an energetically improved gaseous substrate. The integration has two benefits, (i) displacing the air feed to the cement production process with O₂ from the electrolysis process, increases the composition of CO₂ in the exit gas of the cement production process, and (ii) the blending of hydrogen produced by the electrolysis process with the CO₂ rich gas produced provides a CO₂ and H₂ gas stream suitable for fermentation processes.

In an embodiment, at least a first portion of the CO₂ from the cement production process and a first portion of the hydrogen from the electrolysis process may be provided to the CO₂ to CO conversion system to produce CO by the following stoichiometric reaction:

CO₂+H₂↔CO+H₂O

The CO produced by the CO₂ to CO conversion system, may be blended with a second portion of the CO₂ derived from the industrial gas stream and a second portion of the produced hydrogen to provide a fermentation substrate having a desired composition. The desired composition of the fermentation substrate will vary depending on the desired fermentation product of the fermentation reaction. For ethanol production, for example, the desired composition may be determined by the following formula:

$$( x ) {H}_{2} + ( y ) CO + ( \frac{x - 2 y}{3} ) {CO}_{2} \to ( \frac{x + y}{6} ) {C}_{2} {H}_{5} OH + ( \frac{x - y}{2} ) {H}_{2} O ,$$

where x>2y for CO₂ consumption. In certain embodiments, the fermentation substrate may have a H₂:CO ratio of less than 20:1 or less than 15:1 or less than 10:1 or less than 8:1 or less than 5:1 or less than 3:1 with CO₂ available in at least stoichiometric amounts according to algebraic formula.

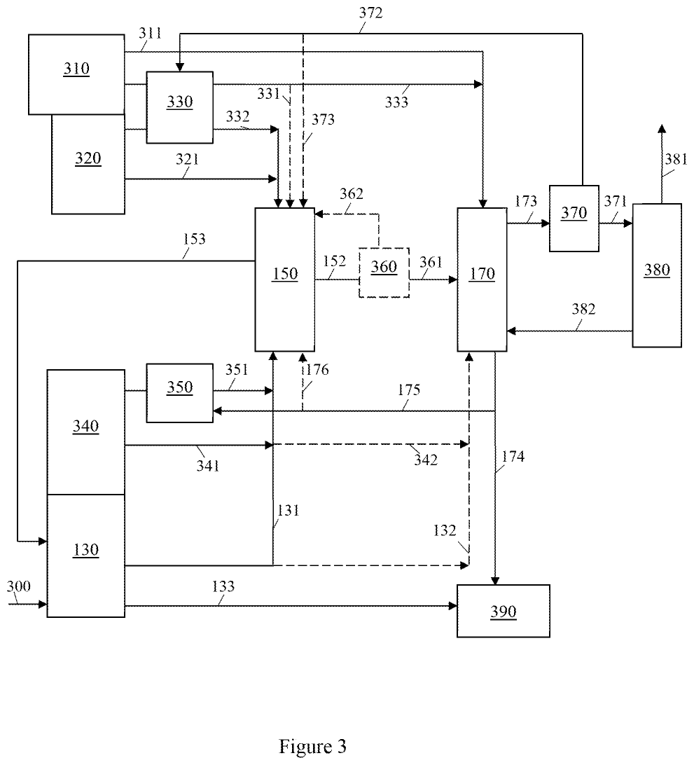

FIG. 3 shows a process integration scheme of one embodiment of the disclosure depicting integration of one or more industrial processes with a CO₂ to CO conversion system, an electrolysis unit, and a gas fermentation process. In FIG. 3, first gas stream comprising CO and H₂ is obtained from industrial process 310. A second gas stream comprising CO₂ is obtained from industrial process 320. A third gas stream comprising H₂ is obtained from industrial process 340. H₂O electrolysis unit 130 receives an energy input 300 to produce a fourth gas stream comprising H₂ and a fifth gas stream comprising O₂. The energy input may be derived from a renewable energy source. Exemplary sources for the renewable energy include, but are not limited to wind power, hydropower, solar energy, geothermal energy, nuclear energy and combinations thereof.

A first portion of the first gas stream and a first portion of the second gas stream are passed to first gas treatment unit 330, to obtain a treated first gas stream and a treated second gas stream. A first portion of the third gas stream is passed to a second gas treatment unit 350 to get a treated third gas stream. The treated second gas stream 332, a second portion of the second gas stream 321, the treated third gas stream 351, a second portion of the third gas stream 341, and optionally a first portion of the treated first gas stream 331, a first portion of fourth gas stream 131 are passed to CO₂ to CO conversion system 150 to produce a gaseous feed stream comprising CO and an output stream comprising H₂O. The output stream 153 is recycled to the H₂O electrolysis unit 130. Optionally the gaseous feed stream 152 is passed to third gas treatment unit 360 to obtain a treated gaseous feed stream and unreacted gas stream comprising unreacted H₂ or CO₂. Optionally the unreacted gas stream 362 is passed to CO₂ to CO conversion system 150. The treated gaseous feed stream 361, a second portion of the first gas stream 311, a second portion of the treated first gas stream 333, optionally a second portion of the third gas stream 342 and optionally a second portion of the fourth gas stream 132 are passed to gas fermentation bioreactor unit 170 to produce a gas fermentation stream and a tail gas stream comprising H₂. The gas fermentation stream 173 is passed to a degasser unit 370 to obtain a product stream comprising at least one fermentation product and CO₂. A first portion of the product stream 371 is passed to vacuum distillation unit 380 to separate into at least one fermentation product 381 and an exit gas stream. The vacuum distillation unit 380 is designed so as to effectively remove product stream from the fermentation broth. The first gas treatment unit, the second gas treatment unit and the third gas treatment unit may comprise a sulfur removal module. The CO₂ to CO conversion system is selected from reverse water gas reaction system, a thermo-catalytic conversion system, partial combustion system, or plasma conversion system

A second portion of product stream 372 is passed to the first gas treatment unit 330. Optionally, a second portion of the product stream 373 is passed to the CO₂ to CO conversion system 150. A first portion of the tail gas stream 175, is passed to the second gas treatment unit 350. Optionally a second portion of the tail gas stream 176 is passed to the CO₂ to CO conversion system 150. A third portion of the tail gas stream 174 and the fifth gas stream 133 are passed to oxidizer unit 390 for air pollution control.

In an embodiment, the oxidizer unit is selected from a thermal oxidizer unit, a thermal reformer unit, a combined heat and power unit and a syngas generation unit. One or more industrial processes is selected from a syngas emitting industrial process, a CO₂ emitting industrial process and a H₂ emitting industrial process. The one or more industrial process may be selected from carbohydrate fermentation, gas fermentation, cement making, pulp and paper making, steel making, oil refining, petrochemical production, coke production, anaerobic digestion, aerobic digestion, natural gas extraction, oil extraction, geological reservoirs, metallurgical processes, refinement of aluminium, copper and or ferroalloys, for production of aluminium, copper, and or ferroalloys, or any combination thereof; or the synthesis gas process is selected from gasification of gasification of coal, gasification of refinery residues, gasification of biomass, gasification of lignocellulosic material, black liquor gasification, gasification of municipal solid waste, gasification of industrial solid waste, gasification of sewerage, gasification of sludge from wastewater treatment, reforming of natural gas, reforming of biogas, reforming of landfill gas or any combination thereof.

In particular embodiments, the one or more industrial process may be a steel manufacturing process selected from basic oxygen furnace, blast furnace and coke oven processes. Coke oven gas (COG) has a typical composition of 5-10% CO, 55% H₂, 3-5% CO₂, 10% N₂ and 25% CH₄. The typical composition of blast furnace (BF) gas is 20-35% CO, 2-4% H₂, 20-30% CO₂ and 50-60% N₂. A typical basic oxygen furnace (BOF) gas comprises 50-70% CO, 15-25% CO₂, 15-25% N₂ and 1-5% H₂.

The substrate and/or C1-carbon source may be syngas, such as syngas obtained by gasification of coal or refinery residues, gasification of biomass or lignocellulosic material, or reforming of natural gas. In another embodiment, the syngas may be obtained from the gasification of municipal solid waste or industrial solid waste.

The composition of the substrate may have a significant impact on the efficiency and/or cost of the reaction. For example, the presence of O₂ may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

The composition of the C1-containing gaseous substrate may vary according to factors including the type of industrial process used, and the feedstock provided to the industrial process. Not all C1-containing gaseous substrates produced will have an ideal gas composition for a fermentation process. Combining the C1-containing gases with a renewable hydrogen stream, an additional CO stream or converting CO₂ in the C1 substrate to CO, provides an energetically improved blended gas stream.

Operating the fermentation process in the presence of hydrogen, has the added benefit of reducing the amount of CO₂ produced by the fermentation process. For example, a gaseous substrate comprising minimal H₂, will typically produce ethanol and CO₂ by the following stoichiometry [6CO+3H₂O→C₂H₅OH+4CO₂]. As the amount of hydrogen utilized by the C1 fixing bacterium increase, the amount of CO₂ produced decreases [e.g., 2CO+4H₂→C₂H₅OH+H₂O]. The general form of the equation is:

$$( x ) {H}_{2} + ( y ) CO + ( \frac{x - 2 y}{3} ) {CO}_{2} \to ( \frac{x + y}{6} ) {C}_{2} {H}_{5} OH + ( \frac{x - y}{2} ) {H}_{2} O ,$$

where x>2y to achieve CO₂ consumption.

When CO is the sole carbon and energy source for ethanol production, a portion of the carbon is lost to CO₂ as follows:

6CO+3H₂O→C₂H₅OH+4CO₂ (ΔG°=−224.90 kJ/mol ethanol)

In these cases, where a substantial amount of carbon is being diverted to CO₂, it is desirable to pass the CO₂ either back to the industrial process, such as a gasification process, or alternatively to send the CO₂ to the CO₂ to CO conversion system. In accordance with the present disclosure, when a CO₂ electrolyzer is present, the CO₂ tail gas may be recycled to the electrolyzer for reduction to CO and O₂.

As the amount of H₂ available in the substrate increases, the amount of CO₂ produced decreases. At a stoichiometric ratio of 1:2 (CO/H₂), CO₂ production is completely avoided.

5CO+1H₂+2H₂O→1C₂H₅OH+3CO₂ (ΔG°=−204.80 kJ/mol ethanol)

4CO+2H₂+1H₂O→1C₂H₅OH+2CO₂ (ΔG°=−184.70 kJ/mol ethanol)

3CO+3H₂→1C₂H₅OH+1CO₂ (ΔG°=−164.60 kJ/mol ethanol)

In a fermentation, where CO₂ is the carbon source and H₂ is the electron source, the stoichiometry is as follows

2CO₂+6H₂→C₂H₅OH+3H₂O (ΔG°=−104.30 kJ/mol ethanol)

The O₂ by-product of the electrolysis production process may be used in the industrial process for the production of the CO₂ gas. In the case of complete oxidation processes, the O₂ by-product of the electrolysis would replace the air feed typically required. Addition of oxygen rather than air increases the composition of CO₂ in the exit gas of the process. For example, a 100% oxygen fed: CH₄+2O₂→CO₂+2H₂O provides 100% CO₂ concentration in the exit gas; whereas air fed: CH₄+2O₂+7.5 N₂→CO₂+2H₂O+7.5 N₂ provides 12% CO₂ in the exit gas stream.

The CO₂ feedstock may be combined with hydrogen produced by electrolysis to provide an optimized feedstock for a CO₂ and H₂ fermentation process. For example, 6H₂+2 CO₂→C₂H₅OH+3H₂O.

The C1-fixing bacterium is typically an anaerobic bacterium selected from of carboxydotrophs, autotrophs, acetogens, and ethanologens. More particularly the C1-fixing bacterium is selected from the genus Clostridium. In particular embodiments, the C1-fixing bacterium is selected from the group consisting of Clostridium autoethanogenum, Clostridium ljungdahlii, and Clostridium ragsdalei.

All references, including publications, patent applications, and patents, cited herein are hereby incorporated by reference to the same extent as if each reference were individually and specifically indicated to be incorporated by reference and were set forth in its entirety herein. Mention of any reference in this specification is not, and should not be taken as, an acknowledgement that that reference forms part of the common general knowledge in the field of endeavour in any country.

The use of the terms “a” and “an” and “the” and similar referents in the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. The use of the alternative (e.g., “or”) should be understood to mean either one, both, or any combination thereof of the alternatives. As used herein, the term “about” means±20% of the indicated range, value, or structure, unless otherwise indicated.

The recitation of ranges of values herein is merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein may be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Multiple embodiments are described herein. Variations of those embodiments may become apparent to those of ordinary skill in the art upon reading the foregoing description. Skilled artisans may employ such variations as appropriate, and it is intended for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. An integrated fermentation and industrial process for improving carbon capture efficiency, the process comprising:
a) converting water in a H₂O electrolysis unit and generating a hydrogen stream comprising H₂;
b) passing at least a portion of a tail gas stream comprising CO₂ from an industrial process to a CO₂ to CO conversion system to produce a gaseous feed stream comprising CO;
c) passing the gaseous feed stream to a gas fermentation bioreactor unit comprising a culture of at least one C1-fixing microorganism;
d) passing at least a portion of the hydrogen stream to the CO₂ to CO conversion system, to the gas fermentation bioreactor unit, or to both;
e) fermenting the culture to produce one or more fermentation products and an exit gas stream comprising CO₂; and
f) recycling the exit gas stream to the CO₂ to CO conversion unit.

2. The process of claim 1 further comprising passing a feedstock comprising CO₂ to a CO₂ electrolysis unit and generating an oxygen stream comprising O₂ and a CO stream comprising CO and passing the oxygen stream to the industrial process and the CO stream to gas fermentation bioreactor unit.

3. The process of claim 2 wherein the CO₂ electrolysis unit and/or H₂O electrolysis unit requires an energy input, wherein the energy input is derived from a renewable energy source.

4. The process of claim 1 wherein the industrial process is selected from a partial oxidation process, a gasification process, a complete oxidation process, or any combination thereof.

5. The process of claim 2 further comprising passing at least a portion of the tail gas stream to a treatment unit to generate a treated tail gas stream and recycling the treated tail gas stream to the CO₂ electrolysis unit.

6. The process of claim 1 wherein the CO₂ to CO conversion system is selected from reverse water gas reaction system, thermo-catalytic conversion system, partial combustion system, plasma conversion system, or any combination thereof.

7. The process of claim 1 wherein the C1-fixing microorganism is selected from Clostridium autoethanogenum, Clostridium ljungdahlii, Clostridium ragsdalei, or any combination thereof.

8. An integrated system comprising;
a) a CO₂ electrolysis unit having a first gas stream outlet and a second gas stream outlet;
b) an industrial process zone comprising an inlet and a tail gas outlet, the inlet in fluid communication with the first gas stream outlet of the CO₂ electrolysis unit;
c) a CO₂ to CO conversion system comprising a feed stream outlet, the CO₂ to CO conversion system in fluid communication with the tail gas outlet;
d) a gas fermentation bioreactor unit comprising a product stream outlet, the gas fermentation bioreactor unit in fluid communication with the feed stream outlet and with the second gas stream outlet; and
e) a H₂O electrolysis unit having a third gas stream outlet wherein the third gas stream outlet is in fluid communication with the CO₂ to CO conversion system, the gas fermentation bioreactor unit, or both.

9. The system of claim 8 wherein the CO₂ electrolysis unit and/or H₂O electrolysis unit is further in electrical communication with a renewable energy production unit.

10. The system of claim 8 wherein the industrial process zone is selected from a partial oxidation process zone, a gasification process zone, a complete oxidation process zone, or any combination thereof.

11. The system of claim 8 wherein the gas fermentation bioreactor unit further comprises an exit gas stream outlet in fluid communication with the CO₂ electrolysis unit.

12. The system of claim 8 wherein the CO₂ to CO conversion system is selected from reverse water gas reaction system, thermo-catalytic conversion system, partial combustion system, plasma conversion system or any combination thereof.

13. The system of claim 8 further comprising a treatment unit in fluid communication with the tail gas outlet and the CO₂ electrolysis unit.

14. An integrated fermentation and industrial process, comprising:
a) obtaining a first gas stream comprising CO and H₂, a second gas stream comprising CO₂ and a third gas stream comprising H₂ from one or more industrial processes;
b) passing an energy input to a H₂O electrolysis unit to obtain a fourth gas stream comprising H₂ and a fifth gas stream comprising O₂;
c) passing a first portion of the first gas stream, and a first portion of the second gas stream to a first gas treatment unit, and a first portion of the third gas stream to a second gas treatment unit to obtain a treated first gas stream, a treated second gas stream and a treated third gas stream;
d) passing a second portion of the second gas stream, the treated second gas stream, a second portion of the third gas stream, the treated third gas stream, a first portion of the fourth gas stream and optionally a first portion of the treated first gas stream, to a CO₂ to CO conversion system to produce a gaseous feed stream comprising CO and an output stream comprising H₂O;
e) passing the output stream to the H₂O electrolysis unit;
f) optionally passing the gaseous feed stream to a third gas treatment unit to obtain a treated gaseous feed stream;
g) passing the treated gaseous feed stream, a second portion of the first gas stream, a second portion of the treated first gas stream, optionally a second portion of the third gas stream and optionally a second portion of the fourth gas stream to a gas fermentation bioreactor unit to produce a gas fermentation stream and a tail gas stream comprising H₂;
h) passing the gas fermentation stream to a degassing unit to obtain a product stream comprising at least one fermentation product and CO₂;
i) passing a first portion of the product stream to a vacuum distillation unit and separating into at least one fermentation product and an exit gas stream comprising CO₂;
j) passing a second portion of the product stream to the first gas treatment unit and optionally a third portion of the product stream to the CO₂ to CO conversion system;
k) passing the exit gas stream to the gas fermentation bioreactor unit;
l) passing a first portion of the tail gas stream to the second gas treatment unit and optionally passing a second portion of the tail gas stream to the CO₂ to CO conversion system; and
m) passing a third portion of the tail gas stream and the fifth gas stream to an oxidizer unit.

15. The process of claim 14 wherein one or more industrial processes is selected from a syngas emitting industrial process, a CO₂ emitting industrial process, a H₂ emitting industrial process, or any combination thereof.

16. The process of claim 15 wherein the industrial process is selected from carbohydrate fermentation, gas fermentation, cement making, pulp and paper making, steel making, oil refining, petrochemical production, coke production, anaerobic digestion, aerobic digestion, natural gas extraction, oil extraction, geological reservoirs, metallurgical processes, refinement of aluminium, copper and or ferroalloys, for production of aluminium, copper, and or ferroalloys, or any combination thereof; or the synthesis gas process is selected from gasification of gasification of coal, gasification of refinery residues, gasification of biomass, gasification of lignocellulosic material, black liquor gasification, gasification of municipal solid waste, gasification of industrial solid waste, gasification of sewerage, gasification of sludge from wastewater treatment, reforming of natural gas, reforming of biogas, reforming of landfill gas, or any combination thereof.

17. The process of claim 14 wherein the energy input is derived from a renewable energy source.

18. The process of claim 14 wherein the first gas treatment unit, the second gas treatment unit, and the third gas treatment unit, comprise a sulfur removal module.

19. The process of claim 14 wherein the CO₂ to CO conversion system is selected from reverse water gas reaction system, a thermo-catalytic conversion system, partial combustion system, or plasma conversion system.

20. The process of claim 14, wherein the oxidizer unit is selected from a thermal oxidizer unit, a thermal reformer unit, a combined heat and power unit or a syngas generation unit.
